(19) United States
(12) Patent Application Publication
(10) Pub. No.: US 2005/0042598 A1
(43) Pub. Date: Feb. 24, 2005

(54) Method for identifying interaction partners using phage display

(76) Inventors: Oliver Hill, Neckarsteinach (DE); Holger Ottleben, Berlin (DE)

Correspondence Address: FOLEY AND LARDNER;SUITE 500, 3000 K STREET NW, WASHINGTON, DC, 20007, US

(21) Appl. No.: 10/496,012

(22) PCT Filed: Nov. 27, 2002
(86) PCT No.: PCT/EP02/13395

(30) Foreign Application Priority Data
Nov. 28, 2001 (DE) 101-58-242.0

(51) Int. Cl. C12Q 1/70

(52) U.S. Cl. 435/5

(57) ABSTRACT

A method for identifying/selecting interaction partners which interact with a target molecule, with the aid of a support to which anchor molecules are applied that form a polymer-free surface to which affinity ligands are covalently bonded, said ligands being brought into contact with viruses that have a plurality of peptides or proteins as interaction partners on their surface is described. The method described guarantees an accumulation of viruses which present specific interaction partners by an optionally cyclic repetition of the selection. Optionally, selected specific interaction partners are expressed recombinantly once the coding nucleotide has been identified. Furthermore, a surface and its use for a phage display method are described.

The present invention relates to a method for identifying/selecting interaction partners which interact with a target molecule with the aid of a support to which anchor molecules are applied that form a surface which is polymer-free and to which affinity ligands are covalently bonded which are contacted with viruses presenting a plurality of peptides or proteins as interaction partners on their surface. The method described guarantees an accumulation of viruses which present specific interaction partners by an optionally cyclic repetition of the selection. According to the invention, optionally, selected specific interaction partners are expressed recombinantly once the coding nucleotide sequence has been identified. Moreover, the invention comprises the described surface and its use for a phage display method.

Different documents are cited in the text of this description. The disclosure content of the cited documents (including all manufacturer's descriptions and indications, etc.) is herewith incorporated by reference into the description.

The identification of target molecules has led to the understanding of many biological processes. Thus, the common experimental approaches have up to now been cell-based screening and affinity chromatography. Although both techniques are helpful in discovering target molecules, a method coupling the protein identification with the gene isolation is highly desirable.

Such an approach is followed in a screening system where in vitro gene expression techniques are combined with traditional biochemical approaches such as e.g. affinity chromatography. The functional gene selection is carried out with a direct link between the natural product affinity and the gene structure of the peptidic affinity ligand. In this case, (non-)viral peptides and proteins are used as ligands which are presented on the surface of recombinant viruses.

Usually, phages are used as expression system for desired (non-)viral proteins or peptides for this method. Phages are known to the person skilled in the art as viruses which specifically infect bacteria and which are, thus, not pathogenic for humans. In phage display, genes coding for (non-)viral proteins or peptides are incorporated into the viral genome in such a way that fusion proteins can be generated between the desired (non-)viral protein or peptide and a viral coat protein. In this manner, the fusion protein is presented on the surface of the virus during the replication of the virus in the host.

In a typical phage library for a phage display, a plurality of DNA fragments coding for (non-)viral proteins or peptides, are inserted into the viral genome. In this manner, viral particles are generated which present a plurality of proteins or peptides on their surface. A corresponding phage display library is then contacted with a sample immobilised on a support. In the subsequent washing step, phages presenting fusion proteins binding to the immobilised sample are retained on the support, whereas phages presenting non-interacting fusion proteins are washed away. The bonded phages are eluted and amplified by infection of a host culture. Repeated amplification and selection circles can be necessary to obtain a phage population which binds to the immobilised sample with high affinity. Subsequently, the inserted DNA segments of individual phage clones are sequenced and the amino acid sequences of the binding proteins or peptides are derived therefrom.

An immobilisation of the interaction partners (affinity ligands) is necessary to make the separation of the phage-ligand complexes from the unbound phages possible.

Affinity ligands can be immobilised in a plurality of ways. The corresponding method depends on different factors, such as e.g. on the kind of ligand or support material.

An immobilisation can be carried out covalently or by adsorption. Usually, a protein, very often an antibody is used as affinity ligand. In the prior art, however, the use of peptides or organic molecules as affinity ligands is also described.

For affinity ligands which are proteins, methods are described where these are directly immobilised onto the support material with the aid of passive adsorption.

Commonly, for this purpose, a corresponding support material is used which is made of polymeric plastic (e.g. polystyrene, polyvinyl, latex) and e.g. in the form of microtiter plates, membranes or spherical beads (cross-linked polymers in particle form) (Lowman, Annu. Rev. Biophys. Biomol. Struct 26 (1997), 401-24). Butteroni et al. (J. Mol. Biol. 304 (2000), 529-540) adsorbed, e.g. the Oct-4 protein in the presence of BSA (bovine serum albumin) on microtiter plates.

A known disadvantage of an adsorptive immobilisation of the affinity ligand is that the polymers used as support materials have a very high unspecific protein adsorption due to their hydrophobic properties. Thus, the free protein binding sites on the support have to be saturated by blocking reagents (e.g. BSA—bovine serum albumin). Biopolymers such as e.g. serum albumin, complex protein mixtures such as protein hydrolysates (gelatin, trypthone), casein hydrolysates, dry milk powder, polyamines and synthetic blocking reagents (e.g. polyvinylpyrrolidone) can be used as blocking reagents. Correspondingly, there is a further disadvantage for such blocking reagents as they, too, have an unspecific protein bond.

Thus, a selection surface which, due to its low unspecific protein adsorption, can avoid the use of blocking reagents, would be more advantageous.

Moreover, it is known that in the case of a direct adsorption of proteins which are used as affinity ligands, these are partially denatured by the direct contact with the support material. Thus, phages can be accumulated in the selection process which specifically bind to the structure formed by the denaturation. Moreover, the immobilisation of the affinity ligands by adsorption takes a long time (several hours). With the above indicated, directly adsorptive immobilisation method, only proteins can be used as affinity ligands for the phage display. This fact markedly reduces the field of application of the phage display technology.

A decrease of the unspecific protein adsorption can be achieved by not directly contacting the protein used as affinity ligand with the support material. To achieve this, anchor molecules are used with the aid of which the proteins are bonded to the support. In this way, the native structure of the protein used as affinity ligand can be preserved.

Correspondingly, in the prior art, the use of antibodies which are directed against the ligand protein and which are adsorbed to the surface, is proposed. The ligand protein is then, according to this method, immobilised by the specific antibody-antigen recognition. The prerequisite for this is that the antibody has a sufficiently high affinity for the ligand protein to be immobilised. Crameri et al. (Eur. J. Biochem. 226 (1994), 53-58) e.g. immobilised monoclonal anti-human-lgE-antibodies in microtiter plates. By adding the sera of patients, the immunoglobulins of the lgE class were bound as affinity ligands for the phage display. Consequently, the fact that, prior to a corresponding method, antibodies against the affinity ligand used have to be generated in a time-consuming and costly manner, is a disadvantage of this method.

The use of anchor molecules moreover makes it particularly possible to immobilise small affinity ligands which, due to their molecular size cannot themselves be immobilised by passive adsorption. Consequently, methods are described according to which the immobilisation of small molecules is carried out by coupling them to proteins which can be immobilised by adsorption. Dörsam et al. (FEBS Letters 414 (1997), 7-13) used steroids covalently bonded to BSA as small affinity ligands.

Other proteins which are very frequently used as anchor molecules are streptavidin and avidin, which non-covalently bind to the ubiquitously occurring biotin (vitamin H) with a high affinity (10⁻¹⁵ M). Here, a biotinylation of the affinity ligand is necessary which then binds to a surface correspondingly coated via streptavidin or avidin. Due to the very slow dissociation rate of the biotin-conjugated molecule, the use of this system for the affinity selection with phage display is possible. (Griffiths and Duncan, Current Opinion in biotechnology 9 (1998), 102-108). Disadvantageous, unspecific interactions have, however, also been described for the biotin-streptavidin/avidin system (e.g. with free sugar groups (Houen and Hansen, Journal of Immunological methods 210 (1997), 115-123). A further disadvantage results from the fact that the bonding of the biotinylated affinity ligand to the surface coated with streptavidin/avidin can be competed by endogenous biotin of the host system.

When ligands are immobilised with the aid of streptavidin or avidin, the phage-ligand complexes are usually competitively separated with biotin from the affinity support. Consequently, biotin is carried over to the subsequent steps of the selection process and has to be removed in a time-consuming manner prior to the next selection circle. This is commonly done by a polyethylene glycol-mediated precipitation of the phages, precipitation and subsequent reintegration in a biotin-free buffer system (Sche et al., Chem. Biology 6 (1999), 707-716; Sche et al., Chem. Biol. 8 (2001), 399-400). It is moreover disadvantageous that the affinity ligand has first to be biotinylated in a time-consuming and costly manner.

It is moreover described that it is a great disadvantage when using proteins as anchor molecules that unspecific protein bonding to these anchor molecules occurs through protein-protein interactions. These unspecific protein bondings can be decreased by blocking reagents. The use of such blocking reagents, however, has the disadvantages described above.

Due to the non-covalent bonding of the affinity ligand to the surface or the support material, all of the immobilisation methods indicated above only make bonding of the affinity ligand possible that can be competed. This can lead to the phage-ligand complexes separating from the surface or the support material and thus not being selectable.

Apart from the use of hydrophobic polymers such as, e.g. polystyrene, the use of hydrophilic polymers such as, e.g. sepharose as support material for the immobilisation of affinity ligands was described. The advantage of hydrophilic polymers is that, due to their hydrophilic properties, they have a lower unspecific protein bonding than hydrophobic polymers. Hydrophilic polymers which are used as support material for the phage selection are natural or chemical modified polysaccharides such as, e.g. dextrane, cellulose and nitrocellulose, cross-linked and/or pearl-shaped agarose (e.g. sepharose), mixed matrices from dextranes and highly cross-linked porous agarose (such as, e.g. superdex), as well as hydro gels from polysaccharides or from linked allyldextrane/N,N-methylene bisacrylamide copolymers.

The bonding of the affinity ligand or of a bond-mediating component (anchor molecule) to these hydrophilic polymers occurs, in this method, usually by covalent coupling. The fact that the affinity ligand is more stably bonded is an advantage of the covalent coupling. The hydroxyl residues of polysaccharides for example can, by specific reagents (e.g. bromocyanogen), be activated in such as way that amine-containing compounds can be bonded covalently (Hermanson, Bioconjugate Techniques, Chapter 2.1, Academic Press, 1996).

Gram et al. (Eur. J. Biochem. 246 (1997), 633-637) describe the immobilisation of Src-homology 2-domains (SH2 domains) of an adaptor protein Grb2 as GST (glutathion-S-transferase) fusion protein to bromocyanogen-activated sepharose beads.

Hawlish et al. (Analytical Biochemistry 293 (2001), 142-145) describe the use of 15 mer peptides which have been synthesised on cellulose membranes as affinity ligands. Here, the initial coupling occurred via a β-Ala-β-Ala dipeptide to the membrane which had previously been activated.

It is an apparent disadvantage of the indicated hydrophilic polymers that they have a three-dimensional structure with cavities. Due to the porous surface, the affinity ligands are not presented uniformly on the surface and are not equally accessible. Other polymers with a porous surface which are used for the phage selection are polyvinylidene fluoride (PVDF), (functionalised) nylon membranes, polyethylene membranes or polypropylene membranes.

In order to avoid the described disadvantage of porous surfaces, polymeric surfaces in the form of solid spherical particles such as, e.g. acrylamide-bisacrylamide-copolymers, polymethacrylate, polyethylene and polypropylene can be used. These solid particles can be piled and used as affinity columns. Generally, interspaces form between the solid spherical particles in which dead volumes of phage suspension can accumulate.

Another disadvantage of the immobilisation of the affinity ligand on the above indicated polymeric surfaces is that due to the density of the ligand which cannot be directly controlled, steric effects between the relatively large phage particles and the polymeric surface and between the protein expressed on the phage or the peptide and the polymeric surface can occur on the surface. This can also lead to the identification of false-positive interaction partners. Determining a density which is optimum for the selection of the affinity ligand on the support material is very time-consuming and costly. In the case of the passive adsorption, e.g. the affinity ligand is diluted in different concentrations in the blocking reagent and is contacted with an unknown number of coupling sites of the support material. Here, a competition for the groups of the support material which mediate the bonding occurs between the affinity ligand and a diluting component contained in the blocking reagent. The result is a surface layer consisting of a mixture of the affinity ligand and accompanying polymeric substances from the blocking reagent, e.g. BSA. In any case, the chemical properties of the surface are determined by the blocking reagent (variation of the chemical properties of the parts of the surface which are not determined by the ligand). Another disadvantage is that a very large amount of affinity ligand is necessary for the diluting series. This is particularly a problem if the affinity ligand is only available in very small amounts and/or is very expensive. Consequently, it is desirable to control the ligand density presented on the surface by the number of binding sites of the support material and not by the concentration of the affinity ligand in the blocking reagent during the adsorption.

A surface where the ligand density can be controlled would, thus, be advantageous. Another disadvantage which has been described with respect to the polymeric surfaces mentioned above is that the regeneration for further selection circles is impossible or very time-consuming. Reagents which make it possible for the regeneration of the surface to occur in a one-step process (e.g. SDS-containing solutions or methanol trifluoro acetic acid mixtures) cannot be used as they change or destroy the structure of the polymers. Moreover, the use of such reagents can optionally lead to the separation of the polymers from the support. The use of proteins as anchor molecules makes it moreover possible to use only mild conditions for the regeneration.

Correspondingly, it would be advantageous to use a surface for the phage selection which can be regenerated in a one-step process.

Thus, the problem underlying the present invention is to provide a method which can be used easily and a surface for a corresponding method for identifying/selecting specific interaction partners, which avoid the disadvantages described above, i.e. for example that the surface should have a low protein adsorption and should be regenerative.

This technical problem is solved by the embodiments characterised in the claims.

Consequently, the present invention relates to a method for the identification/selection of interaction partners which interact with a target molecule by using a support onto which anchor molecules have been applied which form a polymer-free surface and to which affinity ligands are covalently bound, wherein the method comprises the following steps:
(a) contacting the affinity ligands immobilised on the anchor molecules with viruses which present a plurality of peptides or proteins as interaction partners on their surface;
(b) removing unbound viruses from the surface; and
(c) detection of an interaction between the affinity ligands and the interaction partners presented by the viruses.

In connection with the present invention, the terms “identification” and “selection” mean an accumulation, preferably an individualisation of interaction partners. Consequently, the both identification of interaction partners in a large population of any different interaction partners and the selection of individual interaction partners in a population which has previously been accumulated, are comprised.

In the context of the present invention, the term “interaction partner” is used for describing a population of molecules which comprises molecules interacting with other molecules. Thus, the definition of the interaction partners comprises peptides and proteins which show an interaction with a target molecule.

An interaction can, e.g. be characterised by a “key-hole principle”. The interaction partner (peptide or protein) and the target molecule (affinity ligand) have structures or motifs which specifically match, such as e.g. an antigenic determinant (epitope) which interacts with the antigen binding site of an antibody. By knowing the structure of one of the interaction partners, conclusions can be drawn with respect to possibly preferred structures or with respect to special structural elements of a suitable partner which interacts therewith. In the method according to the invention, the interaction partners are presented on the surface of viruses as peptides or proteins. Here, all peptides or proteins are comprised the coding nucleotide sequences of which can be inserted into a viral genome. It is preferred that the expression of these peptides or proteins as part of the virus coat allows an assembly of this coat and thus the propagation of the virus. Preferably, the propagated virus is infectious. The term peptides or proteins comprises both natural and synthetic peptides or proteins. Examples of natural proteins comprise, inter alia, antibodies, antibody fragments, receptors which are able to bind their specific ligands, peptidic ligands which interact with their specific receptors or peptide domains interacting with specific substrates, including proteins and co-enzymes and with other peptides or enzymes etc. Here, recombinantly produced forms of the aforementioned proteins or peptides are also comprised. Correspondingly, natural peptides comprise amongst others fragments of the above described proteins which interact with specific affinity ligands. Synthetic proteins or peptides comprise both pseudogens which were brought to expression or fragments thereof and proteins or peptides with a random amino acid sequence. The peptides and proteins are thus preferably a component of a library consisting of viruses, wherein the viruses, preferably integrated in their genome, contain a nucleic acid sequence which encodes the corresponding peptide or protein. Here, this nucleic acid sequence is typically present in such a way that, upon expression, it leads to the synthesis of the peptide or protein as part of a fusion protein which consists of a coat protein of the virus or of a part thereof and of the peptide or protein. This fusion protein has then the ability of being localised on the surface of the virus and thus presenting the peptide or protein.

In connection with the method of the invention, the term “affinity ligand” describes molecules or compounds which are immobilised by covalent bonding on anchor molecules which are described in detail below. Consequently, according to the invention, the term affinity ligand comprises all substances and compounds which are to be immobilised on these anchor molecules. Thus, in the connection of the present invention, the term “affinity ligand” comprises both organic and inorganic substances and compounds, e.g. also macromolecules and small molecules or low molecular molecules. The term moreover includes both chemically unmodified and modified molecules.

The term “macromolecules” means molecules with a high molecular complexity or high molecular weight. Preferably, these are biomolecules such as e.g. biopolymers, in particular proteins, oligopeptides or polypeptides but also DNA, RNA, oligonucleotides and polynucleotides, prosthetic groups, lipids, oligosaccharides and polysaccharides as well as their modifications and also synthetic molecules. In connection with proteins, particularly receptors are suitable but also proteins or peptides which represent epitopes or antigenic determinants of proteins. Moreover, the proteins can also be fusion proteins.

The term “small molecules” or “low molecular molecules” refers to molecules with lower molecular complexity than the macromolecules defined above. In the literature, the term “small molecules” or “low molecular weight molecules” is not used uniformly. In WO 89/03041 and WO 89/03042, molecules with molecular weights of up to 7,000 g/mol are described as small molecules. Usually, however, molecular weights of between 50 and 3,000 g/mol, more often, however, between 75 and 2,000 g/mol and mostly in the range between 100 and 1,000 g/mol are indicated. Examples are known to the person skilled in the art from documents WO 86/02736, WO 97/31269, U.S. Pat. No. 5,928,868, U.S. Pat. No. 5,242,902, U.S. Pat. No. 5,468,651, U.S. Pat. No. 5,547,853, U.S. Pat. No. 5,616,562, U.S. Pat. No. 5,641,690, U.S. Pat. No. 4,956,303 and U.S. Pat. No. 5,928,643.

Within the present invention, the term “small molecules” is used for molecules (without anchor and without additional molecule) with a molecular weight of between 50 and 3,000 g/mol, preferably between 75 and 1,500 g/mol. As an example of such small molecules, oligomers or also small organic molecules can be indicated such as oligopeptides, oligonucleotides, carbohydrates (glycosides), isoprenoids or lipid structures. In the literature, mostly the molecular weight forms the basis for the definition of such small molecules.

The examples of the present application show that by using the tetrapeptide Ac-pYVNV as affinity ligand from a human liver cDNA expression library (see following examples 1 to 7) three independent bacteriophage clones could be isolated the capsids of which presented human protein fragments with sequence homologies to the SH2-domain.

In connection with the present invention, the term “anchor molecules” describes compounds or molecules which serve as a connection between the affinity ligand and a support. According to the invention, they form a surface on the support which presents the affinity ligands on the side which is not pointing to the support.

Anchor molecules which are suitable for the method of the invention are characterised in that they form a polymer-free surface on the support. “Polymer-free” means that this surface does not comprise polymers.

A polymer can be considered a substance which is formed by a plurality of molecules, in which one kind or several kinds of atoms or atom groupings (constituting units) are repeated sequentially, wherein the physical properties of the substance do not notably change when the number of the components is slightly changed. With respect to polymers, this is, inter alia, the case if their medium molar mass amounts to at least 10,000 g/mol. Moreover, biopolymers such as e.g. polypeptides with at least 50 amino acids are also considered polymers. “Polymer-free” moreover preferably means that the anchor molecules forming the surface are not cross-linked with each other. It was surprisingly found that especially the use of anchor molecules which form polymer-free surfaces is particularly advantageous in a phage display method as they guarantee a lower unspecific protein adsorption of the surface. Preferably, blocking reagents do not have to be used.

The anchor molecules used in the method according to the invention preferably comprise at least two functional moieties which are situated at opposite ends of the anchor molecule. One of these functional moieties serves as a link of the anchor molecules with the support (anchor group), the other immobilises the affinity ligands. Hereinafter, the latter is also referred to as “head group”. Accordingly, the immobilisation of an affinity ligand means the bonding of this ligand to the head group of an anchor molecule. Such an anchor molecule is moreover characterised in that it is applied with its anchor group onto a support.

Affinity ligands are covalently bonded to the head group of the anchor molecules. The following example 1 describes an example of an immobilisation of affinity ligands by covalent bonding to anchor molecules.

It is an advantage of the described bonding of the affinity ligands to the anchor molecules that the polymer-free surface which is used for the selection can easily be regenerated. In the context of the present invention, regeneration means the complete removal of phages or proteins bonded to the surface. To this avail, reagents which make a regeneration of the surface in a one-step method possible (e.g. SDS-containing solutions, formic acid or methanol trifluoroacetic acid mixtures) can be used. One-step methods can only be used in a limited way for the regeneration of the polymeric surfaces used in the prior art as the reagents used in this connection change or destroy the structure of the polymers, or can lead to the separation of the polymer from the support. With respect to the polymer-free surface used in the method of the invention, however, the selection of the regeneration substances is only limited as regards the stability of the affinity ligand. In the context of the present invention the term “support” describes all phases onto which a surface which is suitable for the method of the invention can be applied. These phases are preferably solid phases. Examples of supports which are suitable for the method of the invention comprise solid supports consisting of one or several parts. The parts are moreover preferably characterised in that they comprise materials selected from the group consisting of glass, plastic, natural or synthetic membrane, metal, metal oxide or non-metal oxide.

The application of the polymer-free surface onto the support using the described anchor molecules can be carried out in one step or in several steps.

In the case of a one-step method, the interaction partner/affinity ligand is linked to the head group of the anchor molecule prior to its immobilisation. Suitable anchor molecules for the one-step method are described in WO 00/73796 A2. Here, in order to provide a measurement surface, a complete ligand-anchor molecule conjugate (LAK) is applied onto the surface. This is a molecule which links the affinity ligand to be immobilised and the functional group necessary for application onto the surface by means of a structure which is able to form an SAM bond.

In the case of multiple-step methods, e.g. first a bonding surface in the form of a organic border layer is generated which does not yet present the desired specific structure characteristics. It is, however, characterised in that the affinity ligands can be bonded to it directly or after their activation. In order to immobilise the affinity ligand, it is, e.g. linked only after the production of a first border layer, which has no bonding specificity, by a covalent bonding defined above via the head group of the anchor molecules. To this avail, the head group can be used directly or after activation. A selection of such head groups is described in EP 485 874 A2.

The application of the affinity ligand to be immobilised is not limited to specific methods. For the localisation of the active sites on the bonding surface e.g. common pipetting or spot apparatuses but also stamp or ink-jet methods can be used.

Techniques for a described contacting of the affinity ligands which are immobilised on the anchor molecules with viruses which present a plurality of peptides or proteins as interaction partners on their surface are known to the person skilled in the art. Moreover, a possible method is described in annexed example 3. Further conditions for the contacting are described in the prior art e.g. in T7Select® System Manual, Fa. Novagen, Madison (USA), (TB178 June 2000), p. 12 seqq.

In a preferred embodiment of the invention the anchor molecules form a highly ordered, self-assembled molecular monolayer or are part of such a monolayer. As a result, self-organising, highly ordered uniform structures at a phase border form by in general hydrophobic interactions. Examples and preferred embodiments of such anchor molecules are described further below.

In a further preferred embodiment of this method, the anchor molecule on which the affinity ligands are immobilised comprises an additional molecule which enhances the coupling of the affinity ligand to the anchor molecule. In the context of the present invention, the additional molecule is considered to belong to the anchor and has, thus, also to guarantee the property of being polymer-free.

An example of such an additional molecule is cysteine. (For introducing a protected 2-aminoethyl thiol to oligonucleotides cf. D. Gottschling et al. Bioconjugate Chem. 9 (1998), 831-837.)

The additional molecule can, moreover, have the function of a spacer (spacer) between the head group of the anchor molecule and the immobilised affinity ligand, whereby the latter is kept away from the head group and, thus, the electronic and steric properties of the head group do not influence the binding properties of the interaction partner.

In a further, also preferred embodiment of the invention, the surface comprises diluting molecules in addition to the anchor molecules.

The diluting molecules at the surface guarantee that the spatial vicinity of adjacent ligands can be controlled. This is achieved by not only applying anchor molecules onto the support but that these are “diluted”. If, for an interaction analysis, a ligand is bound to the surface, adjacent ligands can influence each other on the surface or can influence the interaction of the adjacent ligand with the interaction partner to be detected. In order to prevent such a steric hindrance, mixed surfaces are applied which are composed of ligand-carrying anchor molecules and so-called diluting molecules which do not carry ligands and which, in this manner, dilute the density of the anchor molecules on the surface. In the method of the invention, the concentration of the affinity ligand on the surface is preferably exclusively determined by the ratio of anchor to diluting molecules in the surface and not by the concentration of the ligand in the liquid to be applied, as it is carried out in the prior art.

The diluting molecules should preferably have a structure which does not influence the interaction of the affinity ligand with the interaction partner presented on the viruses. Moreover, they are preferably characterised in that no specific or unspecific bonds occur between them and the interaction partners (e.g. diluter with as high a protein adsorption resistance as possible). Moreover, between the anchor molecule and the diluting molecule, there is preferably as close a structural similarity as possible in order to guarantee that their mixing behaviour on the support is as homogenous as possible.

In another preferred embodiment of the method of the invention, the surface concentration of the affinity ligand is adjusted via the ratio of anchor molecules to diluting molecules.

A (diluted) surface concentration of affinity ligands is produced by a chemical reaction of the affinity ligand to be immobilised with the head group of the anchor molecule. For this reason, in a one-step-method, e.g. the ligand-anchor molecule-conjugate has a thiol group. For a two-step-method, e.g., the affinity ligand has a thiol group which reacts with the head group of the anchor molecule (e.g. maleimide). The formation of a covalent bond with the head group of the anchor molecule is as selective and quantitative as possible (cf. Boeckler et al., J. Immun. Meth. 191 (1996), 1-10). If no suitable functional group for these reactions is present in the corresponding molecules, it can be produced by reacting present functional groups in the ligand to be immobilised. An example of this is the reductive cleavage of a disulfide bond to a thiol (Parham, J. Immunol. 131 (1983), 2895-2902). If a support with a gold surface is used, a thiol group can e.g. be used in order to link the anchor molecules (and diluting molecules) to the support.

In another preferred embodiment, the support is divided into individual parts in each of which different affinity ligands are immobilised at the anchor molecules and thus a plurality of different ligands is bound to the respective anchor molecule of a support.

Removing the unbound viruses according to step (b) of the method of the invention can be carried out according to conventional methods which are known to the person skilled in the art. Preferably, the unbound viruses are removed by elution from the surface. According to the “specific interaction” defined above, the term “unbound viruses” comprises viruses which do not specifically interact with the immobilised affinity ligand(s). An elution process is e.g. a washing process. Here, the surface can e.g. be treated with suitable solutions, the composition of which ensures that the specific interaction of the interaction partner with the target molecule is not dissolved. In this connection, elution conditions with a different stringency are also comprised, where less strong but still specific interactions are dissolved and thus an accumulation or identification of highly specific interacting interaction partners takes place. An example of an elution method is described in the following example 3, further examples are known from the prior art, cf. e.g. in T7Select® System Manual, Novagen, Madison (USA) (TB178 June 2000), p. 14 seqq.

In a preferred embodiment, the method of the invention moreover comprises a step (b′) which is effected after step (b):
(b′) propagation of the bound viruses by infection of a host.

Conditions for a propagation of bound viruses by infection of a host are exemplarily described in the attached example 3. However, corresponding conditions are known to the person skilled in the art from the prior art and, inter alia, described in T7Select® System Manual, Novagen, Madison (USA) (TB178 June 2000), p. 18 seqq.

Moreover, the method of the invention comprises a step (b″) which is also preferred and which is carried out after step (b′):
(b″) repeating step (a) with the propagated virus population.

If step (a) is repeated after the propagation of the viruses, a further selection round with an accumulated viral population is carried out.

In a further preferred embodiment of the invention, steps (a) to (b″) are repeated several times before detecting a specific interaction between the affinity ligands and the interaction partners presented by the viruses in step (c).

By repeating steps (a) to (b″), a selected accumulation of viruses is ensured, which present interaction partners of the immobilised affinity ligands on their surface.

The detection of the interaction between the affinity ligands and the interaction partners presented by the viruses according to step (c) of the method of the invention can be carried out according to conventional methods which are known to the person skilled in the art.

In a preferred embodiment of the invention, the detection of the interaction between the affinity ligands and the interaction partners presented by the viruses in step (c) is based on an immunologic, optical, oscillation-based, radioactive or electrical method.

The detection of the affinity ligand-virus-interaction is possible with systems where an indicator system is linked to the interaction partner/virus. This linkage can optionally be carried out in a further, additional process step. Such systems can be based on ELISA (enzyme-linked immunosorbent assay), radioimmunoassay (RIA), surface plasmon resonance (SPR) or comparable methods for measurement. Methods for measurement which are based on optical phenomena are preferred.

SPR measurements are particularly preferred as no marking of the viruses is necessary in this case.

In an embodiment which is moreover preferred, the detection of the specific interaction between the affinity ligands and the interaction partners presented by the viruses in step (c) is carried out without markers.

It is also preferred that the detection of the specific interaction is carried out by an optical reflection. Preferably, the interaction is hereby detected by the determination of the surface plasmon resonance (SPR). As explained above, supports for the method of the invention consist, for instance, of a metal, preferably a precious metal, particularly preferred gold or have a layer of such a metal on their surface. This metal layer can optionally be applied by means of an intermediate layer which serves as adhesion mediator. The material used, onto which the surface is applied, depends on the method of measurement used. If methods with an optical reflection such as surface plasmon resonance (SPR) are used, a support consisting of glass or plastic is preferred. If sulphur-containing compounds are used for the immobilisation of ligands, which is described below, a gold layer is preferably applied onto them and, for adhesion purposes, a chrome layer is applied.

In another preferred embodiment, the method moreover comprises a step (i) which is either carried out after step (b′) and thus prior to another selection round or after step (c):
(i) characterisation of the binding of the selected virus populations and of individual virus clones derived from these virus populations to the affinity ligands used for the selection, in an assay.

Any kind of assay which is suitable for the characterisation of a bond can be used as assay. Preferably, such an assay is a solid phase assay. Examples 4 and 5 describe such a solid phase assay. As other methods which are known in the literature ELISA (enzyme-linked immunosorbent assays), RIA (radioimmunoassay assay) and surface plasmon resonance (SPR) or the oscillation resonance method (Butler, J. E., METHODS 22, 4-23 (2000)) are described.

According to the invention, the characterisation of the bond in step (i) is also carried out on the same or the identical surface on which the virus population and the individual viral clones derived from this virus population were identified/selected.

Due to the method described herein, the use of surfaces with identical properties both for the selection process and for checking of the binding behaviour of the selected viruses e.g. by means of SPR analysis is for the first time possible. Although Houshmand et al. (Anal. Biochem. 268 (1999), 363-370) describe a phage display system where a biosensor analysis is carried out, the surface used for the biosensor analysis, however, differed with respect to its properties from the surface used for the selection.

In a preferred embodiment, the detection of the interaction between the affinity ligands and the interaction partners presented by the viruses (peptides/proteins) in step (c) is carried out on the surface on which the viruses were identified/selected.

The spatial development of the carrier for the use of the method of the invention is not limited. However, for the detection of the interaction on the same surface on which the viruses were identified/selected, in general, a planar, two-dimensionally stretched structure is preferred. Depending on the area of use, optionally three-dimensional forms such as balls or hollow bodies can, however, also be used.

Moreover, a preferred embodiment of the method also comprises step (d):
(d) isolating and sequencing of the DNA segment of individual virus clones coding for the peptide or the protein of the interaction partner.

From the prior art, the person skilled in the art knows suitable methods with the help of which he can after the isolation of individual viral clones, isolate and analyse the DNA sequences inserted therein which encode the corresponding peptides or proteins. A correspondingly suitable method is described in the following example 6.

In another preferred embodiment of the invention, the method furthermore comprises the recombinant expression and isolation of the peptide or protein identified/selected as interaction partner of the affinity ligand.

An example of the recombinant expression of selected fusion proteins is described in the following example 7. From the prior art, the person skilled in the art knows various expression systems and thus further methods for the expression of isolated nucleic acid sequences and for the isolation of the encoded peptides and proteins. These expression systems comprise prokaryotic and eukaryotic systems (cf. in this connection, inter alia, chapter 9.4 Expressionssysteme in: Mühlhardt, Der Experimentator: Molekularbiologie Gustav Fischer Verlag 1999).

In another preferred embodiment, the method of the invention moreover comprises the characterisation of the bond of the recombinantly expressed peptide or proteins to the affinity ligand used for the selection in an assay. Here, preferably the same assays as used for checking the phage clones are used. This preferred embodiment guarantees an optimal comparability of the results. Thus, with the help of corresponding assays e.g. a virus-independent bond of the selected interaction partner can be detected.

In another preferred embodiment of the method, said characterisation is carried out on the same surface which was used for the identification/selection of the corresponding virus.

Anchor molecules for a method of the invention preferably correspond to the general formula
HS—R-M,
wherein R is a structural element, which ensures the formation of an SAM and M represents a mercaptophilic head group.

Accordingly, the residue R preferably represents an unbranched or branched, optionally substituted, saturated or unsaturated hydrocarbon chain, which in turn can optionally be interrupted by heteroatoms, aromates and heterocyclic compounds. The hydrocarbon chain preferably comprises 5 to 150 (chain-) atoms, including heteroatoms. Anchor molecules with structures which make a passive adsorption of the free interaction partner both at the anchor structure and on the measurement surface difficult or avoid it, are particularly preferred.

Suitable structural elements R which further the formation of a monolayer and optionally make the adjustment of suitable spaces of the head groups from the support surface possible with the help of spacer groups are described for the anchor molecules in the printed PCT specification WO 00/73796 A2, the corresponding disclosure content of which is hereby referred to in its entirety as a preferred embodiment of the method of the invention.

Methods for the synthesis of an anchor molecules can be carried out in a solid phase of in a solution. In the case of a representation in a solution, a protective group is preferably added to the thiol group. Suitable protective groups are described in T. W. Greene, P. G. M. Wuts, Protective Groups in Organic Synthesis, Wiley 1999, chapter 6, “Protection for the Thiol Group”. In the case of a solid phase synthesis, the coupling to the resin is preferably carried out via the thiol group, wherein it stays masked during the synthesis. The separation from the solid phase or the protective group takes place in an acidic environment (e.g. 1% TFA in dichloromethane). Thus, side reactions of the thiol with the mercaptophilic group can be avoided.

It is a substantial advantage of the thiol/mercaptophilic group system that under mild reaction conditions, the linking of the interaction partner to be immobilised can take place (room temperature, pH-neutral, buffer solution usable). This is particularly important in the case of instable compounds or proteins which easily denaturated. Another advantage is the fact that compared to carbon acid, amine or amide residues, the thiol group as functionality is only rarely found in active substances. Thus, an undesired reaction between mercaptophilic groups, which possibly remain after the immobilisation of one of the interaction partners, and a target can mostly be avoided.

In particular with respect to the system thiol/maleimide, a high selectivity compared to other functionalities such as hydroxyl, amine, carboxyl or hydroxylic amine groups is moreover known. Another advantage of the formation of the covalent bond is the high reaction rate (cg. Schelté et al, Bioconj. Chem. 11 (2000), 118-123).

In particular, when immobilising a small affinity ligand, e.g. a so-called “small molecule”, the selectivity and the quantitative course of the reaction are of utmost importance for the quantitative assessment of binding studies or interaction analyses. Unlike e.g. in the case of macromolecular recognition structures, here often small differences in the binding intensity of these ligands with the interaction partners presented on the phage have to be dissolved. In this respect, a ligand with a higher affinity which is presented in a lower density (concentration) than another ligand which binds more weakly can lead to a signal which is artificially reduced and, vice-versa, a weaker ligand which is provided in a higher density (concentration) will lead to a signal which is artificially increased with respect to another ligand.

Furthermore preferably, diluting molecules for the method of the invention correspond to the general formula
HS—R—X,
wherein R is a structural element and X is a non-mercaptophilic group.

Here, the overall length of the chain of the diluting molecule is preferably reduced with respect to the anchor molecule, e.g. by one structural unit such as e.g. an ethylene glycol unit. This can e.g. be achieved by leaving out the last building block of the diluting structure in the case of a linear structure of the anchor and of the diluter on commercially available synthesis building blocks. It is particularly preferred that the head group of the diluting molecule differs from that of the anchor molecule. Preferred head groups for the diluting molecule are methoxy and acylamide groups. Acetamide is particularly preferred when using maleimidyl as anchor head group.

For the production of the surface, a solution of the anchor molecules is contacted with the support. If a solution is used which contains a mixture of anchor and diluting molecules in a certain molar ratio, preferred diluted measurement surfaces are obtained.

In a preferred embodiment of the method of the invention, the ratio of anchor molecules to diluting molecules is between 1:2 and 1:10,000.

Diluting ratios which are particularly preferred are in the range of 1:10 to 1:100.

In another preferred embodiment of this method, the overall concentration of anchor molecule to the diluting component is in a range of from 0.001 to 100 mmol/l, particularly preferred at approximately 0.1 to 1 mmol/l. Methods for the production of such a homogenous, binding surface with a large surface are described in the German patent application DE 100 27 397.1. The disclosure content of this patent application is herewith incorporated by reference in its entirety.

It is moreover preferred that the viruses which carry a plurality of interaction partners and which are contacted in step (a) with the affinity ligands are used in different concentrations in solutions.

In another preferred embodiment of the method, the surface on which the affinity ligands are immobilised is organic and thus moreover serves as anchor molecule. Preferably, it comprises both a functional unit for linking the anchor molecules with the support (anchor group) and a functional unit for linking the immobilised affinity ligands (head group).

An exemplary method for the immobilisation of an affinity ligand on a self-assembling monolayer of anchor and diluting molecules is described in example 1. Here, methods are comprised where the affinity ligands are bound directly or are specifically bound to the anchor head groups after their activation.

Moreover, the structural element R preferably comprises a spacer. This spacer makes the adjustment of the overall length of the chain and the flexibility of the ligand-anchor-conjugate possible. A spacer of the structural element R for the method of the invention preferably comprises an unbranched or branched hydrocarbon chain. According to the invention, this hydrocarbon chain can be saturated or unsaturated.

Moreover preferred, a structural element R for the method of the invention has at least two structural subunits R^{a} and R^{b}. Even if only one these subunits is present, preferably R^{a}, however, anchor molecules can already be provided which can be used for the method of the invention. Preferably, R^{a} causes the formation of an SAM. It is also preferred that R^{a} is hydrophobic. It is moreover preferred that R^{a} comprises an unbranched or a branched hydrocarbon chain. Moreover, it is also preferred that the hydrocarbon chains of the structural subunit R^{a} are completely saturated or partly unsaturated.

Preferably, the hydrocarbon chains have 5 to 50 chain atoms, which are optionally interrupted by aromates, heterocycles or heteroatoms. In a preferred form, the hydrocarbon chains correspond to the general formula —(CH₂)_{n—}, wherein n is a natural number. Preferably, this number is 5 to 50, preferably 5 to 25, more preferably 5 to 18 and most preferred 8 to 12.

It is moreover preferred that R^{a} in addition comprises functionalised alkanes. These are characterised in that they carry functional units at their terminal groups which are selected from a group consisting of hydroxylic groups, halogen atoms, carboxylic acid groups and mercapto groups. These terminal functional units facilitate e.g. the linking of the adjacent structural units during the synthesis of the anchor molecules. Necessary components of the anchor, in particular —SH groups can optionally be introduced with their help. It is moreover preferred that these functionalised alkanes are 11-mercaptoundecane acid or their derivatives. In this connection, hydrocarbon chains which are interrupted by heteroatoms, aromates and/or heterocyclic compounds are also preferred.

It is also preferred that the structural element R^{b} comprises a spacer. It is moreover preferred that R^{b} is hydrophilic. It is moreover preferred that, for the method of the invention, R^{b} comprises a hydrocarbon chain. In another preferred embodiment of the invention, this hydrocarbon chain is interrupted by heteroatoms. It is moreover preferred that the chain comprises between 10 and 100 chain atoms. It is also preferred that, in the method of the invention, the structural element R^{b} comprises an oligoether or an oligoamide. The oligoamide is preferably formed of dicarboxylic acids and/or amino carboxylic acids and diamines.

In an also preferred embodiment, the oligoether corresponds to the general formula
—(O-Alk)_{y}-
wherein y is a natural number and Alk is an alkylene residue. It is preferred that y has a value of between 1 to 50, more preferably between 1 to 20 and most preferred between 2 to 10. The alkylene residue has preferably 1 to 20, more preferably 2 to 10 and particularly preferred 2 to 5 C-atoms.

Preferably, the alkylene group or the amines in the method of the invention comprise, independently 1 to 20 C-atoms, more preferably 2 to 10 and most preferably 2 to 5 C-atoms. It is moreover preferred that the structural element R^{b} comprises groups which are interrupted by further heteroatoms, including O-atoms.

In another preferred embodiment, the oligoether corresponds to the general formula
—(O—C₂H₄)_{y}—
wherein y is a natural number.

In another preferred embodiment of the invention, the natural number y is 1 to 10.

For particularly preferred embodiments of R^{b}, it is referred to German patent application DE 100 27 397.1 the disclosure content of which is herewith completely incorporated by reference.

In another preferred embodiment of the method of the invention, the mercaptophilic head group M is selected from the group consisting of
(a) iodo-acetamides and bromo-acetamides;
(b) pyridyl dithio compounds;
(c) Michael acceptors;
(d) acrylic acid derivatives;
(e) esters, amides, lactones or lactams;
(f) methylene-gem-difluorocyclopropanes;
(g) α,β-unsaturated aldehydes and ketones; and
(h) α,β-unsaturated sulfones and sulfonamides.

Preferably, the head group M corresponds to the general formula 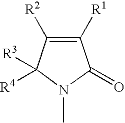
wherein R¹ to R⁴ are independently hydrogen, or C₁ to C₅ alkyl groups or R³ and R⁴ are together ═O. Preferably, R¹ and R² are independently methyl, ethyl or n-propyl. It is also preferred that the mercaptophilic head group M corresponds to a maleimidyl group.

In a preferred embodiment, the interaction partners presented by the viruses are encoded by DNA fragments that are inserted into the virus genome and that form a DNA library. In this case, the number of fragments contained in the DNA library preferably is at least 10³, more preferably at least 10⁴, in particular at least 10⁵, even more preferably 10⁶ and most preferably at least 10⁷.

In another preferred embodiment of the method, the DNA fragments inserted are isolated from a cDNA or genomic DNA (gDNA) or are synthetic oligonucleotides or polynucleotides. It is furthermore preferred that the inserted cDNA or the inserted gDNA is derived from a prokaryotic or eukaryotic organism.

In this case, the eukaryotic organism is preferably a fungus, a plant or an animal organism, preferably a mammal. The mammal is preferred to be a mouse, a rat or a human.

In another preferred embodiment of the method, the cDNA is isolated from a differentiated tissue or a differentiated cell population. In this case, the cDNA is preferably isolated from liver, brain, heart or breast tissue or cells. In this case, the tissues or cells are preferably derived from a healthy organism.

In an alternative preferred embodiment, the tissues or cells are from an unhealthy organism. Preferably, the disease or the ailment of the organism is selected from the group consisting of cancer, hypertrophy and inflammation.

In a preferred embodiment, the viral system comprises a virus that uses eukaryotes as a host.

In another preferred embodiment, the viral system comprises a virus that uses prokaryotes as a host.

In a preferred embodiment of the method, the virus is selected from the group of viruses having double-stranded DNA (dsDNA viruses). It is furthermore preferred that said dsDNA virus is selected from the group of phages. Moreover, it is preferred that the phage is selected from the group of phages with a tail, more preferably from the group consisting of Myoviridae, Podoviridae or Siphoviridae.

In another preferred embodiment of the method, the phage is a bacteriophage specific for Escherichia coli.

In an alternative preferred embodiment of the method, the virus is selected from the group of viruses having single-stranded DNA (ssDNA viruses).

A viral system which is a lytic phage is also preferred. Preferably, said lytic phage has a polyhedral, in particular an icosaedric capsid.

In a preferred embodiment of the method, the lytic phage is a λ phage, a T3 phage, a T4 phage or a T7 phage.

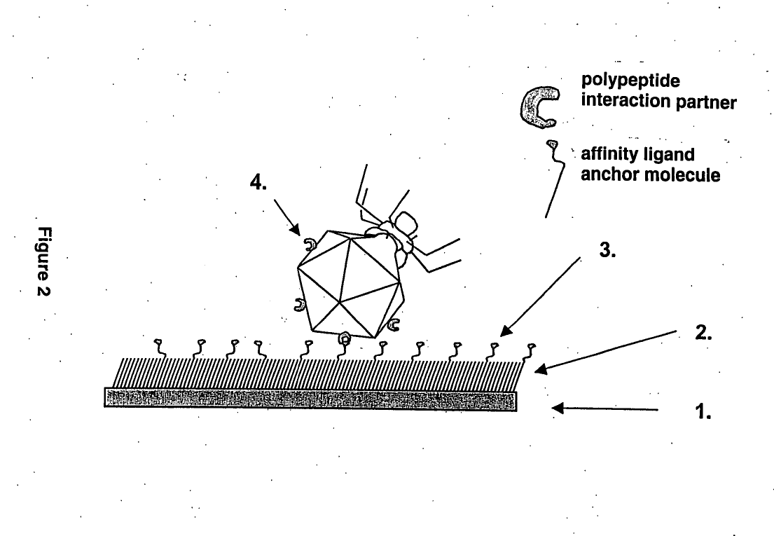

The invention described moreover comprises a support onto which a surface used in the above-mentioned methods is applied. Said surface is characterised in that it is free of polymers and comprises compounds (anchor molecules) to which affinity ligands are covalently bonded. Said surface is furthermore characterised in that viruses are bound to the affinity ligands immobilised on said surface. Said viruses present the peptides or proteins on their surface as specific interaction partners of the affinity ligands which specifically interact with the affinity ligands bound to the surface (cf. FIG. 2).

Said support of the invention is preferred to be characterised in that the virus which presents peptides or proteins as interaction partners on the surface and which is specifically bound to the affinity ligands is bound to a host.

The same which has already been said in connection with the method of the invention also applies to the preferred embodiments of the support, the surface, the affinity ligands, the interaction partners and the viruses.

In addition, the invention described comprises the use of a support onto which a surface is applied that is free of polymers and comprises anchor molecules to which affinity ligands are covalently bonded for a method of the invention, preferably for a method of phage display.

The Figures show

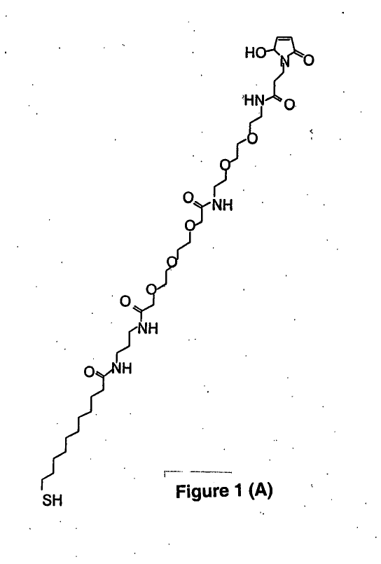

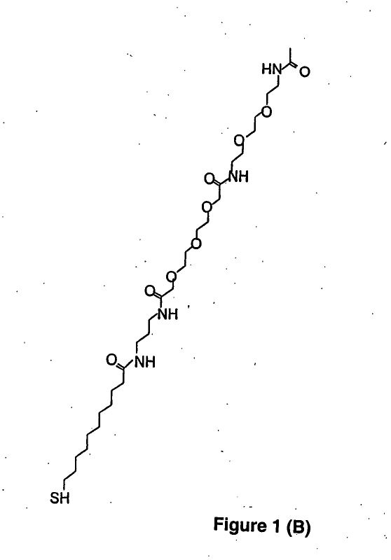

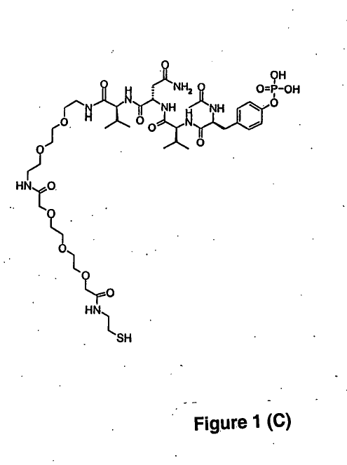

FIG. 1: Structural formulae of the compounds used for producing a surface free of polymers

The Figure shows the anchor molecule (A) used for the self-assembling monolayer (SAM) and the diluting component (B) belonging thereto as well as the affinity ligand (C) which is coupled to the surface later on and which has been chemically modified in such a way that an intermediate molecule is attached to the N-terminus of the ligand which enables coupling to the head group of the anchor by addition of the thiol function to the double-bond of the maleimidyl group. Within the scope of the present invention, intermediate molecules, too, are regarded as part of the anchor and, thus, they must also have the property of being free of polymers.

FIG. 2: Schematic demonstration of the interaction between the polypeptide exposed on the viral envelope and the immobilised affinity ligand

A self-assembling monolayer of anchor molecules and diluting molecules (2) is applied to a solid support (1). An affinity ligand (3) is covalently bonded to the anchor molecules. The virus is bound to the surface formed on the solid support by the anchor molecules and diluting molecules via the interaction partner (4) that is exposed on the viral envelope.

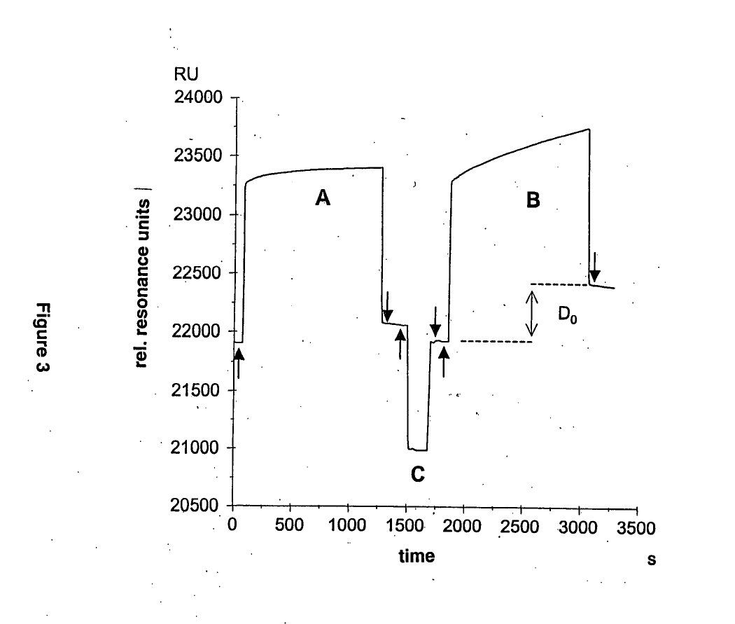

FIG. 3: Sensogram of the binding of different phage lysates to the Ac-pYVNV sensor surface

The binding reactions were detected at a flow rate of 10 μl/min and a temperature of 25° C. HBS buffer (10 mM HEPES; 150 mM NaCl; 3 mM EDTA; 0.001% Tween 20, pH 7.4) was used as a running agent. 200 ml each of the lysates diluted in HBS were conducted over the surface. (A) control lysate: D₀=174 RU; (B) preparative lysate of the forth round of selection: D₀=498 RU; (C) SDS pulse (20 μl 0.5% SDS in water) for the regeneration of the sensor surface. In addition, as an example in measurement B, the D₀ value (difference of the resonance signals before/after injection of the analyte) has been marked. The points in time (beginning/end of the injection) are marked with arrows.

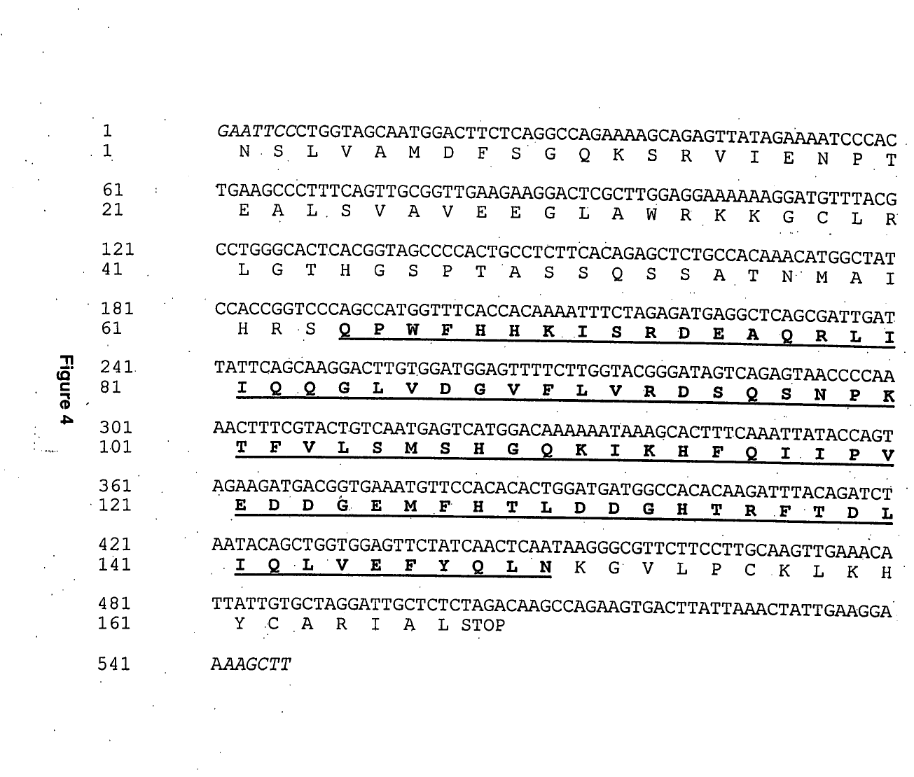

FIG. 4: Sequence of the cDNA insertion from the phages A4-28; A4-30; A4-40 in 5′-3′ orientation of the T710A gene used as a fusion partner

Flanking vector sequences are written in italics. The translation product resulting from the reading frame of the envelope protein is shown below the DNA sequence in a one-letter code. It corresponds to amino acids 174-340 of the human GRB14 protein. The SH2 homologous sequence segment (Src homology domain 2) is underlined and additionally marked in bold letters.

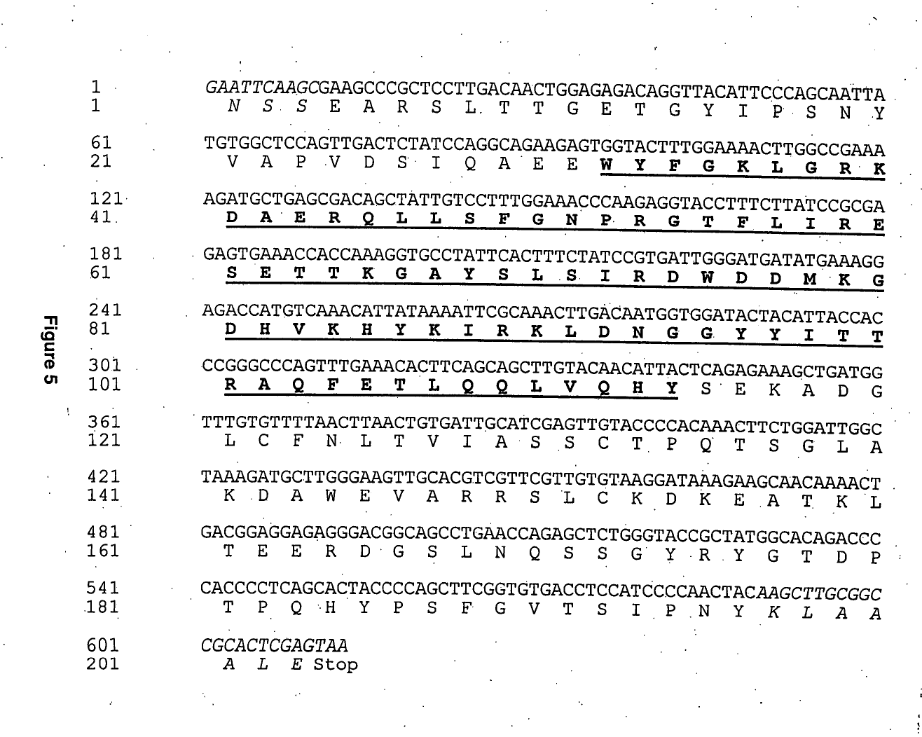

FIG. 5: Sequence of the cDNA insertion from the phages A4-26; A4-39 in 5′-3′ orientation of the T710A gene used as a fusion partner

Flanking vector sequences are written in italics. The translation product resulting from the reading frame of the envelope protein is shown below the DNA sequence in a one-letter code. It corresponds to amino acids 121-268 and 6-50 of the p59fyn(T)=OKT3-induced calcium influx regulator. The SH2 homologous sequence segment (Src homology domain 2) is underlined and additionally marked in bold letters.

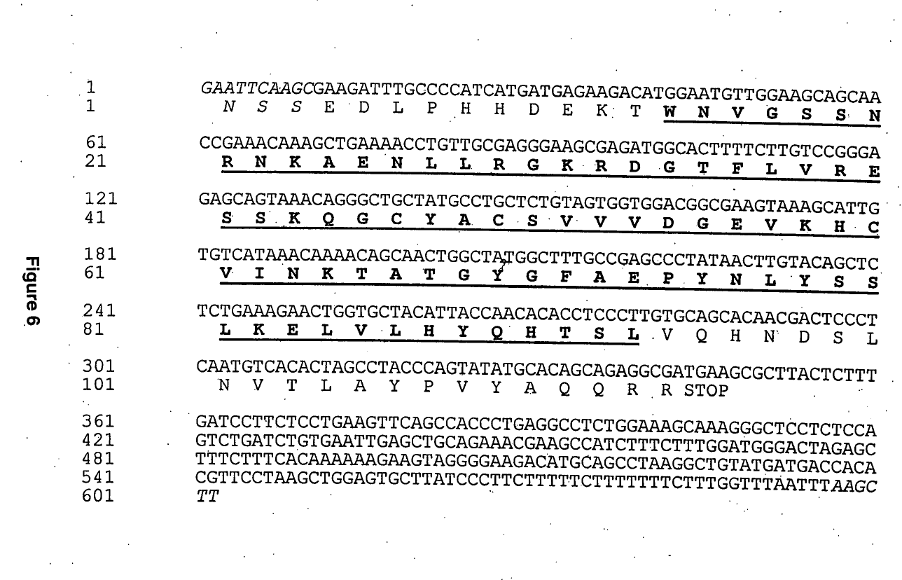

FIG. 6: Sequence of the cDNA insertion from the phages A4-37 in 5′-3′ orientation of the T710A gene used as a fusion partner

Flanking vector sequences are written in italics. The translation product resulting from the reading frame of the envelope protein is shown below the DNA sequence in a one-letter code. It corresponds to amino acids 614-724 of the human phosphoinositide-3-kinase, regulatory subunit, polypeptide 1 (p85 alpha) (PIK3R1). The SH2 homologous sequence segment (Src homology domain 2) is underlined and additionally marked in bold letters.

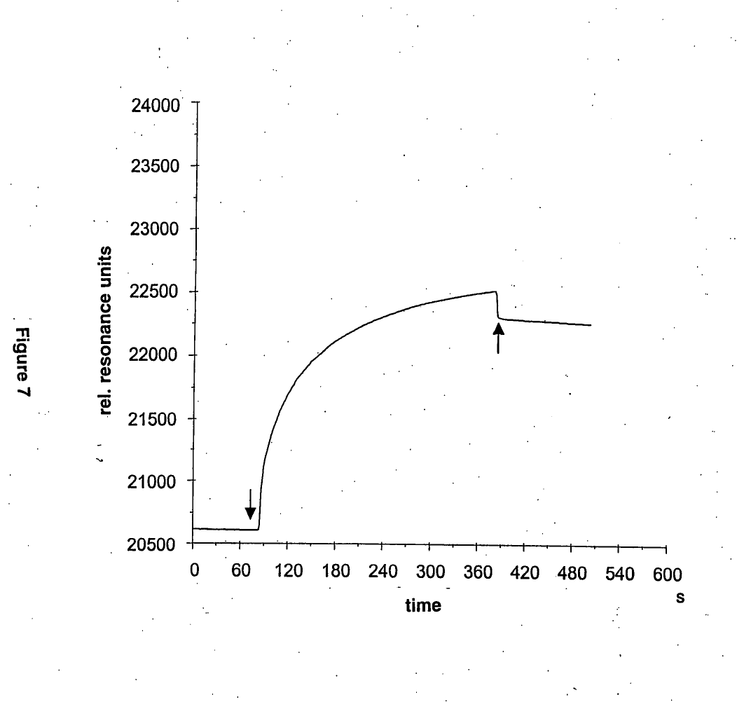

FIG. 7: Sensogram of the binding of the recombinant p59Fyn-SH2 domain to the Ac-pYVNV sensor surface

The binding reactions were detected at a flow rate of 10 μl/min and a temperature of 25° C. HBS buffer (10 mM HEPES; 150 mM NaCl; 3 mM EDTA; 0.001% Tween 20) were used as a running agent. 100 μl of the recombinant protein (c=1 μM) diluted in HBS was passed over the surface. D₀=1673 RU. The points in time (beginning/end of the injection) are marked with arrows.

The following examples illustrate the invention described.

### EXAMPLE 1

### Immobilisation of the Affinity Ligand

For the immobilisation of the affinity ligand, gold surfaces on planar glass supports (hereinafter called chips) were incubated with a mixture of anchor molecule (1 mM) and diluting molecule (1 mM) (cf. FIGS. 1A and 1B), dissolved in methanol:trifluoroacetic acid (99:1 v/v), at a ratio of 1:25 (v/v), hereinafter called coating solution for 60 minutes at room temperature for preparing the self-assembling organic monolayer. Then, the surfaces were washed three times with a mixture of methanol:trifluoroacetic acid (TFA) (99:1) for removing excess coating solution. Subsequently, another washing step was carried out with 0.1% (v/v) TFA in water. Then, washing was carried out with 1 mM acetic acid in water, and the surfaces were dried in a nitrogen stream. For the covalent bonding, a 40 μM solution of the affinity ligand Ac-pYVNV (cf. FIG. 1C) in 200 mM phosphate buffer (pH 7.0) was prepared and the coated gold surfaces were incubated for five minutes in this solution. The excess reagent was removed by washing the chips three times in 20 mM phosphate buffer (pH 7.0). The surfaces were then dried again in a nitrogen stream and stored at 4° C. until further use.

### EXAMPLE 2

### Multiplication of the Phage-cDNA Library

For the biopanning, a commercially available human cDNA phage expression library based on the bacteriophage T7 was used (T7Select™ Human Liver cDNA-Library; NOVAGEN, Madison (USA); Lot no. N14930). The number of primary clones was 1.7×10⁷ pfu (plaque forming units). mRNA from human liver tissue was used as a source for the cDNA fragments cloned into this library. The double-stranded cDNA fragments that were used for cloning and that were synthesised by means of random priming from mRNA were fractionated according to their size (300-3,000 bp) and cloned into the 3′ terminus of the main envelope protein gene (T7-10A gene) of the phage. The directional cloning pattern used by the company makes sure that the cDNA fragments inserted have a quantitative sense orientation to the T7-10A gene and that, thus, the reading direction of the acceptor and donor gene is identical. This results in a larger number of phage clones having human protein fragments as fusion partners on the capsid. An assembly of intact viruses from the fusion protein alone is not possible due to steric inhibition by the fusion partner. The wild-type capsid protein which is located on a plasmid in a particular host strain and is expressed by the virus after infection of the cell is necessary for the formation of intact viral particles.

For obtaining a lysate that is suitable for the selection, 50 ml LB-Amp medium (10 g/l casein hydrolysate, 5 g/l yeast extract, 5 g/l NaCl, 100 μg/ml ampicillin in H₂O, pH 7.4) were inoculated with a single colony of the host strain (E. coli BLT5403) and the cells were propagated aerobically at 37° C. and the culture was infected in the log phase at an OD600=0.32 with 3×10⁸ pfu of the phage library. In the case of an OD600=0.32, there are about 1.2×10⁸ cfu (colony forming units)/ml in the culture; therefore, altogether 6×10⁹ cells were infected; the MOI (multiplicity of infection; number of viruses/number of host cells) was 0.05. A low MOI was of advantage for the multiplication of the cDNA expression library in a liquid culture as it reduced a negative selection of slowly multiplying phage clones by the limitation of the multiplication cycles. Immediately after lysis of the culture, protease inhibitors (final concentration is as follows: 2 μg/ml leupeptin; 1 mM PMSF (phenylmethylsulfonyl fluoride); 1 mM EDTA (ethylene diamine tretra acetic acid) were added and the cell residues were sedimented (10,000×g/15 min/4° C.). Glycerol (10%, v/v) was then added to the supernatant which was then titrated and stored at −80° C. until use in the selection process. The titres of the lysate produced in this manner was 8.6×10⁹ pfu/ml.

### EXAMPLE 3

### Affinity Selection of the cDNA Phage Expression Library

For the first round of selection, 6 ml of the cDNA library lysate (5.16×10¹⁰ pfu altogether) were filled to 20 ml with HBS buffer (10 mM HEPES; 150 mM NaCl; 3 mM EDTA; 0.001% Tween 20, pH 7.4) and contacted with a selection surface as described above. It was incubated for 20 min at room temperature under slight agitation. Then, the phage suspension was removed and the surface was washed seven times with 20 ml TBST buffer (10 mM TRIS, 150 mM NaCl, 0.05% Tween 20, pH 7.4). The remaining volume of the solution that remained on the surface and in the vessel used after washing was determined to be 500 μl beforehand. Subsequently, 1 ml of a host culture growing logarithmically was applied to the selection surface (OD600=0.6-0.8) and left still for ten minutes for infection with the phages bound to the surface. The cells infected were removed mechanically by pipetting and diluted in 50 ml of a logarithmically growing LB-Amp culture of the host strain (OD600=0.3). After dilution, a part of the culture was immediately taken and the number of the cells infected was titrated. Growing was continued until complete lysis of the culture. Immediately after lysis of the culture, protease inhibitors (final concentration as follows: 2 μg/ml leupeptin; 1 mM PMSF; 1 mM EDTA) were added and the cell residues were sedimented (10,000×g/15 min/4° C.). Glycerol was then added to the supernatant (10%, v/v) and stored at −80° C. until use in the second round of selection.

For the second round of selection, 10 ml of the preparative lysate from the selection was filled with HBS buffer to 20 ml and contacted with a new selection surface. The preparation of the chip, the washing steps and the multiplication of the phages was carried out as described in Example 1. Altogether, four rounds of selection were carried out.

The following table shows the number of phages used in each of the rounds of selection (input pfu) and the number of infection events that took place on the selection surface (output pfu) after washing as well as the virus titres of the preparative lysates.

TABLE 1
control phage input/output

| | Selection: | | | |
|---|---|---|---|---|
| | I. | II. | III. | IV. |
| Input pfu: | 5.0 × 10¹⁰ | 2.0 × 10¹¹ | 5.2 × 10¹¹ | 6.0 × 10¹¹ |
| Infected cells | 2.5 × 10⁴ | 2.52 × 10⁵ | 1.47 × 10⁶ | 6.75 × 10⁵ |
| (output pfu): | | | | |
| Titre of the | 2.0 × 10¹⁰ | 5.2 × 10¹⁰ | 6.0 × 10¹⁰ | 4.2 × 10¹⁰ |
| preparative lysate: | | | | |

### EXAMPLE 4

### Checking of the Binding Behaviour of the Lysates Using SPR Measurement

The binding behaviour of the selected phages compared to the immobilised affinity ligand was checked by means of detection of the surface plasmon resonance in a BIACORE® 3000 device (Biacore AB, Uppsala, Sweden). For this purpose, a Biacore Pioneer J1 chip was used.

The affinity ligand was immobilised as described in Example 1.

The different phage suspensions of the selection process served as analytes. From the subsequent time-dependent measurement of the surface plasmon resonance, it was possible to derive immediately the relative affinity of the selected phages compared to the immobilised ligands. For this purpose, the difference between the resonance signal after the end of the injection of the analyte and the resonance signal before the injection of the analyte (D₀ value) was calculated and compared to the corresponding control measurement (negative control) (cf. FIG. 3). Phage suspensions with binding partners had an increased D₀ value compared to the negative control.

Beforehand, the titres of the lysates to be analysed were determined in order to guarantee as identical a number of phages as possible per measurement. Usually, the titres of the preparative lysates obtained in the screening were between 2×10¹⁰ and 6×10¹⁰ pfu/ml (cf. Table 1). Lysates with phage titres in this magnitude were considered to be comparable. The phages were bound to the measurement surface at a constant flow rate. All measurements were carried out on the same Ac-pYVNV chip. Regeneration of the surface between the measurements was carried out by means of SDS denaturation of the phages/ligand interaction. For this purpose, 20-30 μl 0.5% (w/v) SDS (sodium dodecyl sulphate) was injected into water.

TABLE 2
Results of the SPR measurements with the preparative
lysates of the primary selection

| Lysate: | D₀ (RU) | titre (pfu) |
|---|---|---|
| Selection I. | 205 | 2.0 × 10¹⁰ |
| Selection II. | 179 | 5.2 × 10¹⁰ |
| Selection III. | 210 | 6.0 × 10¹⁰ |
| Selection IV. | 498 | 4.2 × 10¹⁰ |
| Control | 174 | 4.3 × 10¹⁰ |

Usually, the phage lysates were diluted 1:10 in HBS buffer and clarified in the centrifuge (20,000×g; 4° C., 5 min) immediately before the injection. For measuring the lysates obtained during the selection process, 200 μl of the dilution were injected at a flow rate of 10 μl/min. The lysates were considered to be positive with regard to their binding behaviour if the D₀ value obtained in the measurement was more than 40% higher than the one of the negative control obtained under identical conditions. A phage lysate having initially 79,000 clones was prepared as a negative control from the cDNA library obtained. The titre of the lysate was 4.3×10¹⁰ pfu/ml. FIG. 3 shows exemplarily the analysis of one of the sensograms obtained.

The D₀ value of the preparative lysate of the fourth round of selection suggested a concentration of binding partners by the selection process.

### EXAMPLE 5

### Secondary Selection Process: Selection Through Measurements of Relative Affinity

For being able to assess the selection process with regard to the increase of the binding partners in the phage population, defined lysates were prepared corresponding to the number of clones, i.e. lysates were grown resulting from the infection of a host culture with only 10 individual phage clones of the screening process.

These clones were isolated for cutting out or elutriating single plaques. For this purpose, the titre plates were used which had been prepared for titrating the host cells infected on the surfaces. Every plaque on these plates corresponded to a phage clone which had bound to the chip surface after the selection process and which had led to an infection event.

The combined lysates obtained in this way were characterised as explained in section 4 by means of SPR measurement with regard to their binding behaviour vis-à-vis the affinity ligand. Deviating from the process described in section 4., the injection volume of the phage suspensions was reduced to 50 μl.

Due to the detection of binding phages after the forth round of selection (cf. Table 2), four phage lysates were prepared as described with 10 clones each from the titre plates of the forth round of selection and measured.

Compared to the control, two of these pools (pool 3 and pool 4) had a positive binding behaviour vis-à-vis the affinity ligand. The 20 phage clones of these two pools were multiplied individually and the lysates were characterised as described using SPR time-dependent measurement as regards their binding behaviour vis-à-vis the Ac-pYVNV sensor surface.

As a result, six clones turned out to be positive binding partners vis-à-vis the affinity ligand.

TABLE 3
Results of the SPR measurements with the preparative lysates
of the defined phage pools from the forth round of selection

| Lysate: | D₀ (RU) | titre (pfu) |
|---|---|---|
| Pool 1. (Clones A4-1 to A4-10) | 92 | 2.9 × 10¹⁰ |
| Pool 2. (Clones A4-11 to A4-20) | 99 | 4.2 × 10¹⁰ |
| Pool 3 (Clones A4-21 to A4-30) | 159 | 5.0 × 10¹⁰ |
| Pool 4 (Clones A4-31 to A4-40) | 203 | 4.5 × 10¹⁰ |
| Control | 75 | 4.3 × 10¹⁰ |

TABLE 4
Results of the SPR measurements of the phage clones
identified as positive binding partners

| Lysate: | D₀ (RU) | titre (pfu) |
|---|---|---|
| Clone-A4-26 | 596 | 5.3 × 10¹⁰ |
| Clone-A4-28 | 334 | 6.6 × 10¹⁰ |
| Clone-A4-30 | 246 | 1.6 × 10¹⁰ |
| Clone-A4-37 | 168 | 2.6 × 10¹⁰ |
| Clone-A4-39 | 396 | 1.6 × 10¹⁰ |
| Clone-A4-40 | 426 | 6.3 × 10¹⁰ |
| Control | 75 | 4.3 × 10¹⁰ |

### EXAMPLE 6

### Analysis of the cDNA Insertions of Positive Individual Clones

From the individual clones identified as positive binding partners in section 5., the sequence segment encoding the fusion portion was amplified by means of PCR from an individual plaque. For this purpose, oligonucleotides were used which hybridise in the vector in regions flanking the passenger DNA. The amplification products were purified, the nucleotide sequence was determined by means of common methods and, first of all, the sequence data obtained were compared. This showed that the cDNA insertions of several phages were identical. Three independent cDNA insertions could be identified in the phage pool sequenced.

Subsequently, the homologous genes were identified by means of BLAST research at the NCBl server:

Insertion I: represented by the clones A4-28; A4-30; A4-40 (cf. FIG. 4). The insertion is partially identical to the sequence of the GenBank entry XM_{—}010770. Definition: Homo sapiens growth factor receptor-bound protein 14 (GRB14), mRNA.

Insertion II: represented by the clones A4-26; A4-39 (cf. FIG. 5). The insertion is partially identical to the sequence of the GenBank entry S74774. Definition: p59fyn(T)=OKT3-induced calcium influx regulator [human, Jurkat J6 T-cell line, mRNA partial, 1605 nt.

Insertion III: represented by the clone A4-37 (cf. FIG. 6). The insertion is partially identical to the sequence of the GenBank entry XM_{—}043864. Definition: Homo sapiens phosphoinositide-3-kinase, regulatory subunit, polypeptide 1 (p85 alpha) (PIK3R1), mRNA.

For identifying known protein domains, a homology search with the translation product of the cloned cDNA sequences resulting from the reading frame of the envelope protein gene was then carried out. Surprisingly, it was found that all cDNA insertions in the reading frame of the envelope protein that had been sequenced encoded human proteins which contained SH2 homologous sequence segments in their primary sequence.

### EXAMPLE 7

### Recombinant Expression of the Fusion Proteins Selected

For proving the specificity of the binding of the proteins identified vis-à-vis the immobilised ligands, the proteins identified were recombinantly expressed in E. coli using conventional methods and purified. Then , the binding behaviour of the recombinant proteins vis-à-vis the selection surface was analysed by means of SPR time-dependent measurement in a Biacore® 3000. All measurements were carried out on the Ac-pYVNV chip which was also used for the analysis of the phage suspensions. FIG. 7 shows exemplarily the binding behaviour of the p59(fyn)-SH2 domain that was expressed recombinantly vis-à-vis the immobilised affinity ligand Ac-pYVNV. The other proteins expressed recombinantly, too, had a positive binding behaviour vis-à-vis the immobilised ligand.

1. A method for the identification/selection of interaction partners which interact with a target molecule by using a support onto which anchor molecules have been applied which form a polymer-free surface and to which affinity ligands are covalently bonded, wherein the method comprises the following steps:
(a) contacting the affinity ligands immobilised on the anchor molecules with viruses which present a plurality of peptides or proteins as interaction partners on their surface;
(b) removing unbound viruses from the surface; and
(c) detecting an interaction between the affinity ligands and the interaction partners presented by the viruses.

2. The method according to claim 1 wherein the anchor molecules form a highly ordered, self-assembled molecular monolayer or are part of such a monolayer.

3. The method according to claim 1 wherein the highly ordered, self-assembled molecular monolayer additionally comprises diluting molecules.

4. The method according to claim 1 wherein the surface concentration of the affinity ligand is adjusted via the ratio of anchor molecules to diluting molecules.

5. The method according to claim 1 wherein the support is divided into individual parts in each of which different affinity ligands are immobilised at the anchor molecules and thus a plurality of different ligands is bound to the respective anchor molecules of a support.

6. The method according to claim 1 wherein the unbound viruses are removed from the surface by elution.

7. The method according to claim 1 further comprising a step (b′) after step (b):
(b′) propagation of the bound viruses by infection of a host.

8. The method according to claim 1 further comprising a step (b″) after step (b′):
(b′) repeating step (a) with the propagated virus population.

9. The method according to claim 8 wherein steps (a) to (b″) are repeated several times before detecting a specific interaction between the affinity ligands and the interaction partners presented by the viruses in step (c).

10. The method according to claim 1 wherein macromolecules are used as affinity ligands.

11. The method according to claim 10 wherein the macromolecules are selected from the group consisting of:
(a) oligopeptides or polypeptides;
(b) oligonucleotides or polynucleotides;
(c) prosthetic groups;
(d) lipids; and
(e) oligosaccharides and polysaccharides.

12. The method according to claim 1 wherein low molecular molecules are used as affinity ligands.

13. The method according to claim 12 wherein the low molecular molecules are inorganic molecules.

14. The method according to claim 12 wherein the low molecular molecules are organic molecules.

15. The method according to claim 1 wherein the detection of the interaction between the affinity ligands and the interaction partners presented by the viruses in step (c) is based on an immunologic, optical, oscillation-based, radioactive or electrical method.

16. The method according to claim 1 wherein the detection of the interaction between the affinity ligands and the interaction partners presented by the viruses in step (c) is carried out without markers.

17. The method according to claim 1 wherein the detection of the interaction between the affinity ligands and the interaction partners presented by the viruses in stet) (c) is carried out by an optical reflection.

18. The method according to claim 17 wherein the detection of the interaction is characterised in that the surface plasmon resonance (SPR) is determined.

19. The method according to claim 1 comprising step (i) which is either carried out after step (b′) or after step (c):
(i) characterisation of the binding of the selected virus populations and of individual virus clones from these virus populations to the affinity ligands used for the selection, in an assay.

20. The method according to claim 19 wherein the characterisation of the binding in step (c) is carried out on the same or the identical surface on which the virus population and the individual virus clones from this virus population were identified/selected.

21. The method according to claim 1 wherein the detection of the interaction between the affinity ligands and the interaction partners presented by the viruses in step (c) is carried out on the surface on which the viruses were identified/selected.

22. The method according to claim 1 comprising step (d):
(d) isolating and sequencing of the DNA segment of individual virus clones coding for the peptide or the protein of the interaction partner.

23. The method according to claim 1 comprising the recombinant expression and isolation of the peptide or protein identified/selected as interaction partner of the affinity ligand.

24. The method according to claim 23 comprising the characterisation of the bond of the recombinantly expressed peptide or protein to the affinity ligand used for the selection, in an assay.

25. The method according to claim 1 wherein the anchor molecules correspond to the general formula
HS—R-M,
wherein R is a structural element which ensures the formation of an SAM and M represents a mercaptophilic head group.

26. The method according to claim 3 wherein the highly ordered, self-assembled molecular monolayer additionally comprises diluting molecules corresponding to the general formula
HS—R—X,
wherein R is a structural element and X is a non-mercaptophilic group.

27. The method according to claim 26 wherein the ratio of anchor molecules to diluting molecules is between 1:2 and 1:10,000.

28. The method according to claim 1 wherein the viruses carrying a plurality of interaction partners, which are contacted with the affinity ligands in step (a), are used in different concentrations in solutions.

29. The method according to claim 1 wherein the surface on which the affinity ligands are immobilised is organic and moreover serves as anchor molecule and which comprises both a functional unit for linking the anchor molecules with the support (anchor group) and a functional unit for linking the immobilised affinity ligands (head group).

30. The method according to any one of claims 25 wherein the structural element R moreover comprises a spacer.

31. The method according to any one of claims 25 wherein the structural element R has at least two structural subunits Ra and Rb.

32. The method according to claim 31 wherein Ra is hydrophobic.

33. The method according to claim 31 wherein the structural element Rb comprises a spacer.

34. The method according to claim 31 wherein Rb is hydrophilic.

35. The method according to claim 25 wherein the head group M corresponds to the general formula 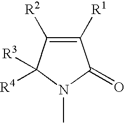
wherein R1 to R4 are independently hydrogen or C1 to C5 alkyl groups or R3 and R4 together represent ═O.

36. The method according to claim 25 wherein the mercaptophilic head group M is a maleimidyl group.

37. The method according to claim 1 wherein the interaction partners presented by the viruses are encoded by DNA fragments which are inserted into the virus genome and which form a DNA library.

38. The method according to claim 37 wherein the number of fragments contained in the DNA library is at least X, wherein X is selected from the group consisting of 103, 104, 105, 106 and 107.

39. The method according to claim 37 wherein the inserted DNA fragments are isolated from cDNA or genomic DNA (gDNA) or are synthetic oligonucleotides or polynucleotides.

40. The method according to claim 39 wherein the inserted cDNA or gDNA is derived from a prokaryotic organism.

41. The method according to claim 39 wherein the inserted cDNA or gDNA is derived from a eukaryotic organism.

42. The method according to claim 41 wherein the eukaryotic organism is a fungus.

43. The method according to claim 41 wherein the eukaryotic organism is a plant or an animal organism.

44. The method according to claim 43 wherein the animal organism is a mammal.

45. The method according to claim 44 wherein the mammal is a mouse, a rat or a human.

46. The method according to claim 44 wherein the cDNA is isolated from a differentiated tissue or a differentiated cell population.

47. The method according to claim 44 wherein the cDNA is isolated from liver, brain, heart or breast tissue or cells.

48. The method according to any one of claims 44 wherein the tissue or the cells are derived from a healthy organism.

49. The method according to any one of claims 44 wherein the tissue or the cells are derived from an unhealthy organism.

50. The method according to claim 49 wherein the disease or the ailment of the organism is selected from the group consisting of cancer, hypertrophy or inflammation.

52-65. (Cancelled)

66. The method according to claim 1 wherein the viral system comprises a virus using eukaryotes as a host.

67. The method according to claim 1 wherein the viral system comprises a virus using prokaryotes as a host.

68. The method according to claim 67 wherein the virus is selected from the group of viruses with a double-stranded DNA (dsDNA viruses).

69. The method according to claim 68 wherein the dsDNA virus is selected from the group of phages.

70. The method according to claim 69 wherein the phage is selected from the group of phages with a tail.

71. The method according to claim 70 wherein the phage is selected from the group consisting of Myoviridae, Podoviridae and Siphoviridae.

72. The method according to claim 69 wherein the phage is a bacteriophage specific for Escherichia coli.

73. The method according to claim 67 wherein the virus is selected from the group of viruses with a single-stranded DNA (ssDNA viruses).

74. The method according to any one of claim 67 wherein the viral system is a lytic phage.

75. The method according to claim 74 wherein the lytic phage possesses an icosaedric capsid.

76. The method according to claim 74 wherein the lytic phage is a λ phage, a T3 phage, a T4 phage or a T7 phage.

77. A support to which a surface has been applied wherein:
(a) the support is free of polymers; and
(b) comprises compounds to which affinity ligands are covalently bound, wherein viruses are bound to the affinity ligands which present peptides or proteins as specific interaction partners of the affinity ligands on their surface.

78. The support according to claim 77 characterised in that the virus which presents peptides or proteins as interaction partners on the surface and which specifically binds to the affinity ligands, is bound to a host.

79. The method according to claim 26 wherein the structural element R comprises a spacer.

80. The method according to claim 26 wherein the structural element R has at least two structural subunits Ra and Rb.

81. The method according to claim 80 wherein Ra is hydrophobic.

82. The method according to claim 80 wherein the structural element Rb comprises a spacer.

83. The method according to claim 80 wherein Rb is hydrophilic.
